(19) United States
(12) Patent Application Publication
(10) Pub. No.: US 2009/0082770 A1
(43) Pub. Date: Mar. 26, 2009

(54) MEDICAL FOOT IMPLANT AND SYSTEM

(75) Inventors: Roland Worner, Tuttlingen-Mohringen (DE); Christoph Schuurmann, Bad Homburg (DE); Uwe Pech, Tuttlingen (DE)

Correspondence Address: BACHMAN & LAPOINTE, P.C., 900 CHAPEL STREET, SUITE 1201, NEW HAVEN, CT, 06510, US

(73) Assignee: NORMED MEDIZIN-TECHNIK VERTRIEBS-GMBH, Tuttlingen (DE)

(21) Appl. No.: 12/236,019

(22) Filed: Sep. 23, 2008

(30) Foreign Application Priority Data
Sep. 25, 2007 (DE) 10 2007 045 885.3

(51) Int. Cl. A61B 17/56; A61F 2/64

(52) U.S. Cl. 606/60; 623/47

(57) ABSTRACT

A medical foot implant (1) with a fastening section (2) for fixing the foot implant (1) to two adjacent bones or bone segments and with a wedge section (3). The wedge section (3) has an end-side tip (12), in a plan view at least approximately punctiform, for easier introduction of the foot implant (1) into bone or into tarsometatarsal joint.

### BACKGROUND OF THE INVENTION

The invention relates to a medical foot implant with a fastening suction for fixing the foot implant to two adjacent bones or bone segments.

In foot surgery a so-called open-wedge operation method is known for example for treating hallus valgus. This operation technique belongs to the environment of conversion osteotomies. The aim of the operation is to change over a joint axis. Changeover takes place in a known manner by at least partial lateral bone severing in which a wedge-shaped osteotomy gap is made. A blunt osteotomy wedge with an elongated, rectangularly contoured end-side (blunt) front side, the purpose of which is to position and fix the bone segments arising from lateral severing at a predetermined wedge angle relative to one another, is introduced into this wedge-shaped gap. The disadvantage to the described method is that the corresponding foot bone must be notched in an upstream severing step at least over part of its diameter prior to the osteotomy wedge being introduced. Not infrequently does this result in unintentional damage to the rear cortical bone.

The object of the invention therefore is to propose an alternative medical foot implant which is configured such that a severing step upstream of introducing the foot implant can be omitted. Another aim is to propose a system for easier introduction of a foot implant.

### SUMMARY OF THE INVENTION

The object is achieved by providing a foot implant with a fastening section including a wedge suction for fixing a foot implant to two adjacent bones or bone segments, the wedge section has an end-side tip, in the plan view at least approximately punctiform, for introduction of the foot implant into bone or into tarsometatarsal joint.

The underlying idea of the invention is to replace the blunt front side in a known osteotomy wedge by a pointed end section. Providing an end-side sharp wedge section allows the medical foot implant (osteotomy wedge) to be forced directly into a foot bone and thus for an upstream severing step during the operation to be at least partially omitted, on the whole accelerating the operation and thus minimizing the strain for the patient. A particular advantage of a medical foot implant designed according to the concept of the invention is that depending on the introduction depth of the medical foot implant in a foot bone extensive shattering of the rear cortical bone of the foot bone can be largely prevented. A medical foot implant designed according to the concept of the invention is suited not only to fixing two adjacent, if needed still partially interconnected or fully separated bone segments at a predetermined wedge angle relative to one another. Due to its form the medical foot implant can also be forced with a pointed wedge section directly into a tarsometatarsal joint of the foot, preferably into a first tarsometatarsal joint, in particular for treating hallus valgus to stiffen the joint and to fix both adjacent bones at a predetermined wedge angle relative to one another. The proposed medical foot implant is preferably (fully) designed from a biocompatible material, preferably titanium or a titanium alloy.

A further development of the invention advantageously provides that the tip of the wedge section in a plan view of the wedge section is at least approximately punctiform. In other words all side sections of the wedge section taper down to a single point, or respectively a punctiform (minimal) area.

Of particular advantage is an embodiment in which the wedge section has four side sections arranged at a right angle to one another, whereby when viewed in the longitudinal direction of the wedge section, each side section tapers in the direction of its respective transverse extension. In other words the wedge section, at least on the end side, preferably has a pyramidal form, whereby the wedge section preferably has a rectangular (if needed notional) base area which is delimited by two long-side pairs of different length. In other words the wedge section has two crossties of varying length, arranged at a right angle to one another.

An embodiment of the foot implant is particularly preferred, in which at least two opposite side sections of the preferably at least selectively pyramidal wedge section are formed curved, preferably concave curved.

Of particular advantage is an embodiment in which at least one opening extending in a transverse direction of the wedge section is provided in the wedge section. Bone material, in particular spongiosa, can grow into this clearance opening or respectively right through it and thus additionally fix the implant.

To secure optimal holding of the foot implant in a foot bone a further development of the invention provides that the circumferential edge of the opening extending in a transverse direction inside the wedge section is not designed smooth but structured.

An embodiment, in which the structuring is designed as serrated structure with teeth arranged adjacent to one another in the peripheral direction of the opening, is particularly preferred, whereby a tooth gap is preferably provided between in each case two teeth extending into the opening.

A further development of the invention advantageously provides that the foot implant is designed such that a forcing aid (forcing pin) can be detachably fixed easily to the latter. The forcing aid can preferably be fixed on the foot implant such that a longitudinal middle axis of the forcing aid aligns with a longitudinal middle axis of the foot implant leading through the end-side tip of the wedge section of the foot implant.

A thread, preferably an inner thread, can be provided for detachable fixing of a forcing aid on the foot implant in a surprisingly simple manner, whereby the thread, in particular the inner thread, is arranged preferably on a side of the foot implant averted from the tip of the wedge section, in particular in its fastening section.

In order to be able to easily fix two bone segments or two adjacent bones relative to one another it is provided according to an advantageous further development of the invention that the fastening section has at least two clearance openings spaced apart from one another for respectively receiving a bone screw, whereby the foot implant with at least one bone screw respectively can be fixed to a bone segment or respectively to a bone.

The clearance openings are preferably arranged in a plate-like region of the foot implant, therefore a plate of the foot implant, which serves as external attachment on the adjacent bones or respectively bone segments. This plate preferably extends transversely to the longitudinal extension of the wedge section.

A further development of the invention advantageously provides that two side sections averted from one another, i.e. arranged on opposite sides, are of varying sizes transversely to their longitudinal extension, thus have a different width extension in each case at a common height, whereby the width extension of both side sections understandably decreases to the punctiform tip, preferably constantly. This produces a nested wedge shape, specifically on one side to the punctiform tip and on the other side in a transverse direction thereto, so as to be able to make an angle correction of the bone segments arranged to the side of the osteotomy wedge in two directions accordingly, one in the direction of forcing and the other transversely thereto.

In other terms, a further development of the invention advantageously provides that two opposite side sections are inclined not only in the direction of their longitudinal extension, i.e. in the direction of the punctiform tip relative to one another, thus arranged at an angle to one another, but also in a transverse direction thereto so as to produce an added wedging effect, specifically transversely to the main wedge direction. In a transverse direction this results in a wedge designed preferably blunt on the end side.

In a further development of the invention it is advantageously provided that the foot implant in two side sections has blind holes extending preferably transversely to the longitudinal extension of the side sections in the direction of the foot implant for a tool to be inserted, by which the foot implant can be removed from the human body or respectively from the bone again.

It should be noted in principle that all side surfaces can have any curved trajectory. An embodiment can accordingly be made in which two opposite side sections are respectively designed concave, or alternatively an embodiment in which two opposite side sections are formed concave and the other two side sections are convex. A combination of two straight (evenly formed) side sections with two concave side sections is equally possible, as well as a combination of two convex formed side sections with two straight side sections.

An embodiment, in which the fastening section, preferably designed as a cover plate, can be fixed detachably on the wedge section in order to be able to utilize a fastening section appropriate or respectively optimized for the respective application, is particularly preferred. Of course, an embodiment can also be made in which the fastening section is designed in one piece with the wedge section.

The invention also promotes a system for easier introduction (forcing) of the medical foot implant into a bone or into a tarsometatarsal joint of the foot. The system comprises a previously described foot implant and a forcing aid which can be fixed detachably on the foot implant, preferably designed as a forcing pin. Particularly preferred is an embodiment in which the forcing aid is provided on the end side with an outer thread, which can be screwed into a preferably centrically arranged inner thread of the foot implant, which extends preferably in a longitudinal direction into the wedge section. After the foot implant is forced into a foot bone or into a joint of the foot the forcing aid can be loosened from the foot implant.

### BRIEF DESCRIPTION OF THE DRAWINGS

Further advantages, characteristics and details of the invention will emerge from the following description of preferred exemplary embodiments and by means of the diagrams, in which:

FIG. 1 shows a perspective illustration of a system comprising a medical foot implant and a forcing aid,

FIG. 2 shows a foot implant in a first side elevation,

FIG. 3 shows the foot implant according to FIG. 2 in a plan view of a fastening section,

FIG. 4 shows a second side view rotated by 90° relative to the FIG. 2 side view of the foot implant,

FIG. 5 is a section illustration of the foot implant along the line of intersection I-I as per FIG. 4,

FIG. 6 shows a perspective illustration of an alternative foot implant with an opening arranged in the wedge section and extending in a transverse direction,

FIG. 7 shows another alternative embodiment of a foot implant without opening in the wedge section, and

FIG. 8 shows another embodiment of a foot implant in a perspective illustration, whereby the illustrated foot implant is configured drop-shaped in a side view, and

FIG. 9a to FIG. 9e are various partially sectional views of a further exemplary embodiment of a foot implant in which two opposite side sections are arranged inclined transversely to their longitudinal extension relative to one another, forming two wedges oriented at 90° to one another.

In the figures the same components having the same function are designated by the same reference numerals.

### DETAILED DESCRIPTION

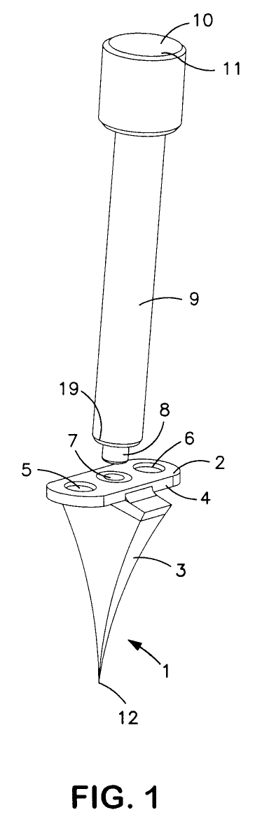

FIG. 1 shows a medical foot implant. The foot implant 1 comprises a fastening section 2 extending transversely to the longitudinal extension of a wedge section 3. The fastening section 2 comprises a plate 4 arched (slightly) transversely to its longitudinal extension, which is fitted with a first and a second clearance opening 5, 6. The longitudinal middle axes of the clearance openings 5, 6 extend parallel to the longitudinal extension of the wedge section 3. The clearance openings 5, 6 respectively serve to receive a bone screw, not illustrated here, for fixing the foot implant from the outside to two adjacent bone segments or bones. As is evident from FIG. 5, the clearance openings 5, 6 are designed chamfered in their upper region for easier finding.

Arranged between the clearance openings 5, 6 spaced transversely to the longitudinal extension of the wedge section 3 is an inner thread 7 which extends through the plate 4 and enters the wedge section 3. The inner thread acts cooperatively with an end-side outer thread 8 of a pin-shaped forcing aid 9. The longitudinal middle axis of the forcing aid 9 at the same time aligns with a longitudinal middle axis of the wedge section 3. The forcing aid 9 is fitted on the front side 10 averted from the foot implant 1 with a circular impact surface 11 for forcing the foot implant 1 into a foot bone or into a tarsometatarsal joint.

In the diagram plane the wedge section 3 which is provided at an end side with a tip 12 which is formed punctiform in a plan view, not shown here, is located under the fastening section 2. The punctiform tip 12 enables forcing into a foot bone or a tarsometatarsal joint without a preceding splitting step. All side sections of the wedge section 3 taper transversely to their respective transverse extension from the fastening section 2 to form this tip 12, viewed in the direction of the tip 12. All four side sections of the wedge section 3 are formed concave curved in the direction of their longitudinal extension.

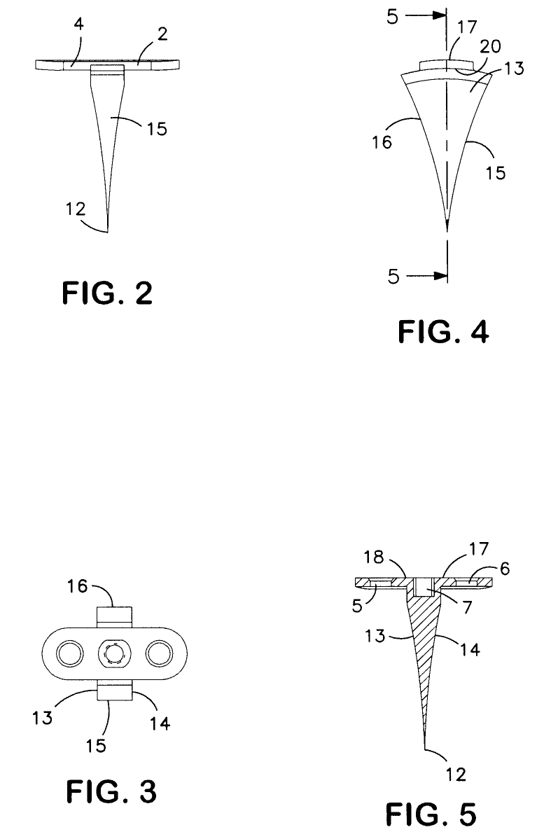

FIGS. 2 to 5 show the foot implant 1 according to FIG. 1 in detail with dimensioning. The dimensioning (dimensions) and corresponding dimension ratios will be deemed disclosed essential to the invention and be claimable. The dimensions and dimension ratios in the diagrams will however not restrict the protective scope of the invention.

As eventuates in particular from FIG. 3, not all side sections 13, 14, 15, 16 have the same width, rather the opposite side sections 13, 14 in every notional sectional plane arranged transversely to the longitudinal extension of the wedge section 3 are broader than the opposite side sections 15, 16 running at a right angle to the latter. As is evident from FIG. 4, the pointed narrower side sections 15, 16 are designed concave curved with a radius of 16 mm. The opposite, broader side sections 13, 14 are formed concave arched by contrast with a radius of 140 mm.

As is evident from FIG. 14 the inner thread 7 is designed to fix the forcing aid 9 shown in FIG. 1 as a metric thread with a diameter of 3 mm (M3). In the region of its upper end in the diagram end the inner thread 7 is enclosed by an annular region 18 which is offset conically relative to the plate top surface 17 and aids in receiving or respectively enclosing a shape-congruent annular cone 19 (cf. FIG. 1) of the forcing aid 9.

As is evident from FIG. 4 the plate top surface 17 is formed arched with a radius of 25.8 mm and the opposite plate underside 20 is formed arched with a radius of 24.5 mm. The plate underside 20 acts as an outer lining on two adjacent foot bone segments or foot bones to be fixed relative to one another.

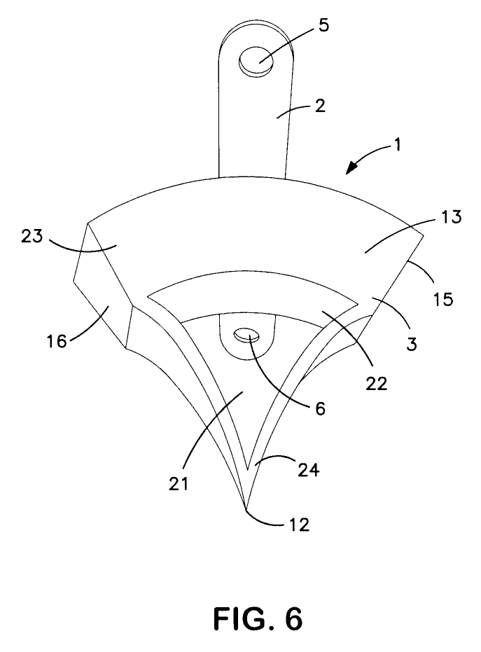

FIG. 6 illustrates an alternative foot implant 1 with a plate-like fastening section 2 and a wedge section 3 bordering it. In the wedge section 3 an opening 21 is provided which joins together both broader side sections 13, 14, and extends in the direction of the least thickness extension of the fastening section 2. The opening 21 matches the form of the fastening section 2. An edge 22 delimiting the opening 21 is designed smooth in the illustrated exemplary embodiment.

Viewed in the longitudinal direction, the fastening section 2 is subdivided into a first upper section 23 and a second, lower section 24 adjoining it in the longitudinal direction, and presenting the tip 12. The sections 23, 24 differ firstly in that the opening 21 is provided exclusively in the second lower section 24. In addition, the side sections 13, 14, 15, 16 are designed concave arched in the second lower section 24, whereas the side sections 13, 14, 15, 16 in the upper first section 23 are designed as even surfaces.

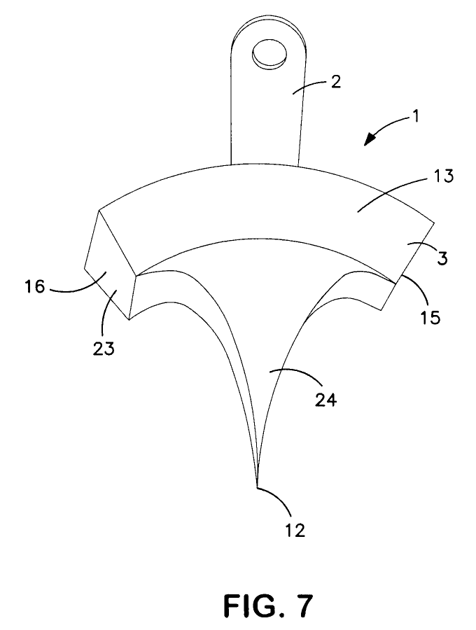

FIG. 7 illustrates a further alternative example of a foot implant 1 with a plate-like fastening section 2 and a wedge section 3, whereby the wedge section 3 can in turn be divided into an upper first section 23 and an adjoining end-side, lower second section 24 with an end-side tip 12 punctiform in plan view. The essential difference to the exemplary embodiment according to FIG. 6 is that in the lower second section 24 no opening running in a transverse direction in the fastening section 2 is provided. As in the exemplary embodiment according to FIG. 6 the side sections 13, 14, 15 in the upper section 23 are designed as even surfaces and in the second lower section 24 are designed as concave curved surfaces.

FIG. 8 illustrates a further exemplary embodiment of a foot implant 1. This comprises a plate-like fastening section 2 with two clearance openings 5, 6 spaced apart in the longitudinal direction of the fastening section 2 respectively for a bone screw. The adjoining wedge section 3 is designed drop-like in a view of one of the broader side sections 13, 14. In the fastening section 2 an opening 21 is provided, the edge 22 of which is formed with teeth 25 spaced apart from one another in the peripheral direction of the opening 21, whereby there is a tooth gap 26 between two teeth 25 adjacent in a peripheral direction with a partially rectangular and partially trapezoidal cross-sectional profile in each case. Also with the exemplary embodiment shown in FIG. 8 the four side sections 13, 14, 15, 16 arranged at a right angle to one another taper in the direction of their transverse extension to the punctiform tip 12.

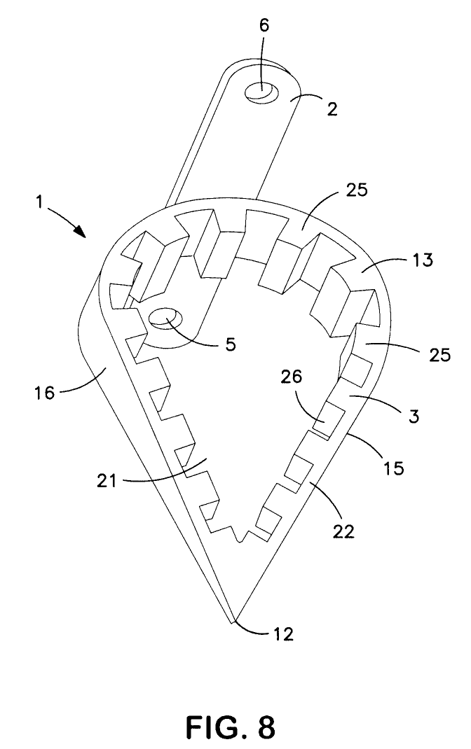

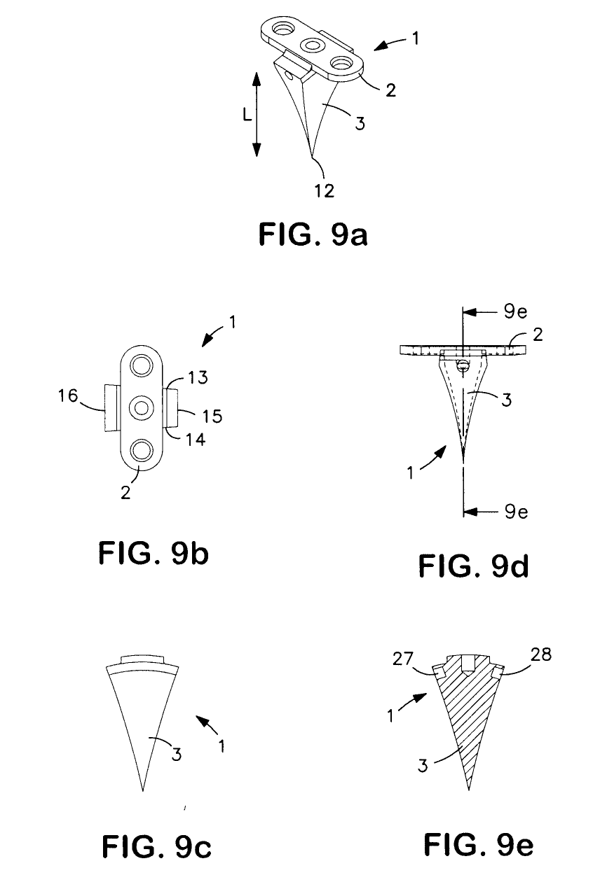

FIGS. 9a to 9e show an alternative exemplary embodiment of a foot implant 1 designed as an osteotomy wedge, which comprises a fastening section 2, which can be fixed detachably to the wedge section 3. The wedge section 3 of the exemplary embodiment shown in FIGS. 9a to 9e differs from all previously described exemplary embodiments in that two opposite side sections 13, 14 are inclined transversely to their longitudinal extension L (cf. FIG. 9a), thus transversely to their extension to the punctiform tip 12, therefore enclosing an angle. The result of this is that both other side sections 15, 16 arranged opposite at a right angle in each case have a different width extension, when viewed at a common height. FIG. 9b shows the maximal width extension of the side sections 15, 16, thus the width extension of the side sections 15, 16 in an upper region. It is evident that the maximal width extension of the side section 16 located to the left in the diagram plane is 2 mm larger than the maximal width extension of the opposite side section 15. From the perspective view according to FIG. 9a it emerges that the side sections 15, 16 of varying width are formed substantially even, whereas the other two side sections 13, 14 inclined to one another in the direction of their longitudinal extension L bulge concave. It is of course possible to fashion any curved form from the different side sections 13, 14, 15, 16.

The sectional view shown in FIG. 9e along the line of intersection A-A according to FIG. 9d shows that a blind hole 27, 28 is made in the straight side sections 15, 16 respectively, whereby the blind holes 27, 28 have a depth extension of 2 mm and enable easier withdrawal of the foot implant 1 from the foot bone.

The osteotomy wedge shown in FIGS. 9a to 9e is suited to three-dimensional correction of bone misalignments, whereby two bone parts to be corrected are aligned by at least two Cartesian coordinate axes.

What is claimed is:

1. Medical foot implant comprising a fastening section including a wedge section for fixing a foot implant to two adjacent bones or bone segments, the wedge section has an end-side tip, in the plan view at least approximately punctiform, for introduction of the foot implant into bone or into tarsometatarsal joint.

2. Foot implant according to claim 1, wherein the wedge section comprises four side sections arranged at a right angle to one another, in each case tapering in a direction of their transverse extension viewed from the fastening section in the direction of the end-side tip.

3. Foot implant according to claim 1, wherein at least two opposite side sections of the wedge section are formed concave in the direction of their respective longitudinal extension.

4. Foot implant according to claim 1, wherein an opening extending in a transverse direction is provided in the wedge section.

5. Foot implant according to claim 4, wherein a structured edge delimits the opening.

6. Foot implant according to claim 5, wherein the structural edge of the opening has a serrated structure with teeth arranged adjacent to one another in a peripheral direction, whereby a tooth gap is provided between adjacent teeth.

7. Foot implant according to claim 1, including fixing means for detachably fixing a forcing aid to the wedge section.

8. Foot implant according to claim 1, wherein the fastening section has a first clearance opening for receiving a first bone screw and at least one second clearance opening spaced apart from the first clearance opening for receiving a second bone screw.

9. Foot implant according to claim 8, wherein the first and second clearance openings are made in a plate extending at least approximately perpendicularly to a longitudinal extension of the wedge section for external attachment to two bones or bone segments.

10. Foot implant according to claim 2, wherein two opposite side sections have a width extension of differing size and the same height.

11. Foot implant according to claim 2, wherein two opposite side sections are inclined to one another transversely to their longitudinal extension.

12. Foot implant according to claim 2, wherein at least one blind hole is made in two opposite side sections.

13. Foot implant according to claim 1, wherein the fastening section is one of (a) one piece with the wedge section and (b) detachably fixed to the wedge section.

14. System comprising a foot implant according to claim 1, further including a forcing aid which can be fixed detachably on the foot implant.
